(19) United States
(12) Patent Application Publication
(10) Pub. No.: US 2009/0208555 A1
(43) Pub. Date: Aug. 20, 2009

(54) CONTROL OF THE DEGRADATION OF BIODEGRADABLE IMPLANTS USING A COATING

(75) Inventors: Marc Kuttler, Berlin (DE); Claus Harder, Uttenreuth (DE); Carsten Momma, Rostock (DE); Heinz Mueller, Erlangen (DE); Daniel Lootz, Rostock (DE)

Correspondence Address: BRYAN CAVE POWELL GOLDSTEIN, ONE ATLANTIC CENTER FOURTEENTH FLOOR, 1201 WEST PEACHTREE STREET NW, ATLANTA, GA, 30309-3488, US

(73) Assignee: Biotronik VI Patent AG

(21) Appl. No.: 10/596,791

(22) PCT Filed: Sep. 7, 2004
(86) PCT No.: PCT/EP2004/010077

§ 371 Date: Mar. 9, 2008

(30) Foreign Application Priority Data
Dec. 24, 2003 (DE) 10361940.2

(51) Int. Cl. A61K 9/00; A61P 9/10

(52) U.S. Cl. 424/426

(57) ABSTRACT

The invention relates to an endovascular implant, which is at least largely biodegradable and whose in vivo degradation can be controlled. To achieve this, the implant comprises a tubular base body, open on its end faces and consisting of at least one biodegradable material, said base body having an in vivo, location-dependent first degradation characteristic D₁(x), in addition to a coating that covers the base body completely or in sections and consists of a biodegradable material, said coating having an in vivo, location-dependent second degradation characteristic D₂(x). According to the invention, a location-dependent cumulative degradation characteristic D(x) in one location (x) is made up of the sum of the respective degradation characteristics D₁(x) and D₂(x) in said location (x) and the location-dependent cumulative degradation characteristic D(x) is predetermined by a variation of the second degradation characteristic D₂(x) in such a way that the degradation in the given location (x) of the implant takes place over a predeterminable time period at a predeterminable degradation rate.

### PRIORITY CLAIM

This patent application is the U.S. National Phase of International Application No. PCT/EP2004/010077, having an International Filing Date of Sep. 7, 2004, which claims priority to German Patent Application No. DE 103 61 940.2, filed Dec. 24, 2003, the disclosures of which are incorporated herein by reference in their entirety.

### FIELD OF THE INVENTION

The present invention relates to an at least predominantly biodegradable endovascular implant, whose in vivo degradation is controllable.

### BACKGROUND OF THE INVENTION

In recent years, the implantation of endovascular support systems has been established as one of the promising therapeutic measures for treating vascular illnesses in medical technology. Thus, for example, in intervention treatment of stable angina pectoris with coronary heart disease, the insertion of stents has resulted in a significant reduction of the rate of restenosis and therefore to improved long-term results. The higher primary lumen gain is the main reason for using stent implantation. By using stents, an optimum vascular cross-section, which is primarily required for therapy success, may be achieved, but the permanent presence of a foreign body of this type initiates a cascade of microbiological processes, which may result in gradual growing over of the stent. One approach for solving these problems is therefore to manufacture the stent from a biodegradable material.

Greatly varying materials are available to medical technicians for implementing biodegradable implants of this type. In addition to numerous polymers, which are frequently of natural origin or are at least based on natural compounds for better biocompatibility, more recently, metallic materials have been favored, because of their mechanical properties, which are significantly more favorable for implants. In this context, materials containing magnesium, iron, and tungsten are considered in particular. One of the objects to be achieved in the practical implementation of biodegradable implants is the degradation characteristic of the implant in vivo. Thus, it is to be ensured that the functionality of the implant is maintained at least over the period of time required for the treatment purposes. In addition, the degradation is to occur as uniformly as possible over the entire implant, so that fragments are not released in an uncontrolled way, which could be the starting point of undesired complications. Known biodegradable stents do not display a locally tailored degradation characteristic.

Proceeding from the related art, it would be desireable to have a biodegradable implant whose degradation may be optimized as a function of location.

This may be achieved by an endovascular implant having the features of:
a tubular main body, open on its front sides, made of at least one biodegradable material, the main body having a location-dependent first degradation characteristic D₁(x) in vivo, and
a coating, which completely or possibly only partially covers the main body, made of at least one biodegradable material, the coating having a location-dependent second degradation characteristic D₂(x) in vivo, and
a location-dependent cumulative degradation characteristic D(x) resulting at a location (x) from the sum of the particular degradation characteristics D₁(x) and D₂(x) existing at the cited location (x) and the location-dependent cumulative degradation characteristic D(x) being predefined by variation of the second degradation characteristic D₂(x) in such way that the degradation at the cited location (x) of the implant occurs in a predefinable time interval having a predefinable degradation curve,

As a result of these features, the degradation characteristic of the entire stent may be locally optimized in the desired way.

### SUMMARY OF THE INVENTION

The present invention accordingly includes the ideas that the degradation of the main body of the implant may be tailored through suitable coating—but also by leaving out the coating in the extreme case—in such a way that the degradation characteristic existing at a location allows a degradation of the implant in a predefinable time interval and having a predefinable degradation curve.

“Biodegradation” is understood to include hydrolytic, enzymatic, and other degradation processes caused by metabolism in the living organism, which result in gradual dissolving of at least large parts of the implant. The term biocorrosion is frequently used as a synonym. The term bioresorption additionally comprises the subsequent resorption of the degradation products.

Materials suitable for the main body may be of polymeric or metallic nature, for example. The main body may also be made of multiple materials. The shared feature of these materials is their biodegradability. Examples of suitable polymer compounds are primarily polymers from the group comprising cellulose, collagen, albumin, casein, polysaccharides (PSAC), polylactide (PLA), poly-L-lactide (PLLA), polyglycol (PGA), poly-D,L-lactide-co-glycolide (PDLLA/PGA), polyhydroxy butyric acid (PHB), polyhydroxy valeric acid (PHV), polyalkylcarbonates, polyorthoester, polyethylenterephthalate (PET), polymalonic acid (PML), polyanhydrides, polyphosphazenes, polyamino acids and their copolymers, as well as hyaluronic acid. Depending on the desired properties of the coating system, the polymers may be provided in pure form, in derivatized form, in the form of blends, or as copolymers.

Metallic biodegradable materials are based on alloys of magnesium, iron, or tungsten. The biodegradable magnesium alloys in particular display an outstandingly favorable degradation behavior, may be processed well, and display no or only slight toxicity, but rather even appear to positively stimulate the healing process.

The main body of a stent is typically assembled from multiple support elements situated in a specific pattern. Depending on the application—whether it is for dilatation or due to the obstruction of the surrounding tissue, for example—the support elements are loaded by different mechanical forces. With biodegradable materials, inter alia, this may result in the areas of the support elements under stress or at least temporarily subjected to high mechanical strains being degraded more rapidly than less stressed areas. Inter alia, the present invention allows this phenomenon to be counteracted.

The coating may also be made of the above-mentioned biodegradable materials. Of course, multiple different materials may also be used in an implant, for example, at different locations or as multilayer systems at a specific location of the implant.

“Location-dependent degradation characteristic” as defined in the present invention is understood to mean the chronological curve (degradation curve) and the time interval in which this degradation occurs. The time of the implantation itself is used as the first reference point for the time interval for the sake of simplicity. Of course, other points in time may also be used. An end of the time interval as defined in the present invention is understood as the time at which at least 80 weight-percent of the biodegradable implant mass has been degraded or the mechanical integrity of the implant no longer exists, i.e., the implant may no longer perform its support function. The degradation curve indicates at what speed the degradation occurs at specific times in the time interval. Thus, for example, through appropriate modifications according to the present invention, the degradation of the implant may be strongly delayed in the first two weeks after the implantation through suitable coating and only progresses continuously after degradation of the coating due to the more rapid degradation of the main body. In order to allow the degradation processes to proceed suitably, it is therefore necessary to know the degradation characteristic of the main body at the specific location of the implant and, in addition, to influence the overall degradation behavior of the implant at this location by applying a coating having a second degradation characteristic. The degradation characteristics of the main body and the coating may be estimated beforehand with the aid of in vitro experiments.

The degradation characteristic of the coating is preferably achieved by
varying its morphological structure,
material modification of the material, and/or
adapting a layer thickness of the coating.

By adapting the layer thickness of the coating, the location-dependent degradation characteristic of the implant may be influenced. Controlling the degradation at a specific location chronologically and in its extent is also in the foreground here. Thus, from a medical viewpoint, it is necessary to maintain the support function of the implant over a specific period of time, and possibly also as a function of location. The degradation of the implant at a specific location may be delayed using an elevated layer thickness.

“Morphological structures” as defined in the present invention are understood as the conformation and aggregation of the compounds forming the coating, particularly polymers. This includes the type of the molecular order structure, the porosity, the surface composition, and other intrinsic properties of the carrier, which influence the degradation behavior of the biodegradable material on which the coating is based. Molecular order structures comprise amorphic, (partially) crystalline, or mesomorphic polymer phases, which may be influenced and/or produced as a function of the particular manufacturing method, coating method, and environmental conditions used. Through targeted variation of the manufacturing and coating methods, the porosity and the surface composition of the coating may be influenced. In general, with increasing porosity of the coating, the degradation occurs more rapidly. Amorphic structures show similar effects to (partially) crystalline structures.

“Material modification” as defined in the present invention is understood to include both derivatization of the biodegradable material, in particular the polymers, and also the addition of fillers and additives for the purpose of influencing the degradation characteristic. Derivatization comprises, for example, measures such as cross-linking or replacing reactive functionalities in these materials. Thus, for example, it is well known that by increasing a degree of cross-linking, polymer materials such as hyaluronic acid are degraded more slowly. These measures must also be first quantitatively detected by established in vitro assays, in order to be able to deliver an estimation of the degradation characteristic for the in vivo behavior.

The location-dependent degradation characteristic of the implant is preferably predefined as a function of pathophysiological and/or rheological conditions to be expected in application. The pathophysiological aspects take into consideration the fact that the stent is typically placed in the vessel in such way that it presses essentially against the lesion, i.e., the adjoining tissue has different compositions at the ends and in the middle area of the stent and therefore the support function of the implant has to be maintained for different periods of time to optimize the healing process. Furthermore, the tissue resistances acting on the implant are unequal because of the pathophysiological change, which may result in a degradation accelerated by the resulting mechanical stress occurring at the locations of stronger resistance.

Rheological aspects in turn take into consideration that the flow conditions are different, particularly in the area of the ends and in the middle sections of the stent. Thus, there may be accelerated degradation of the implant at the ends of the stent because of the stronger flow. Rheological parameters may particularly vary strongly by predefining the stent design and must be determined in the individual case. By considering the two cited parameters, degradation which is optimal for the desired therapy may be ensured over the entire dimension of the stent.

### BRIEF DESCRIPTION OF THE DRAWINGS

The present invention will be explained in greater detail in the following on the basis of exemplary embodiments and in the associated drawings.

FIG. 1 shows a stent having a tubular main body, open at its front sides, whose peripheral wall is covered with a coating system;

FIG. 2a shows a schematic cross-section along a longitudinal axis of a stent to illustrate the coating according to a first variation;

FIG. 2b shows a schematic cross-section along a longitudinal axis of a stent to illustrate the coating according to a second variation;

FIG. 3a shows a schematic cross-section along a longitudinal axis of a stent to illustrate the coating according to a second variation; and

FIG. 3b shows a schematic cross-section along a longitudinal axis of a stent to illustrate the coating according to another variation.

### DESCRIPTION OF THE INVENTION

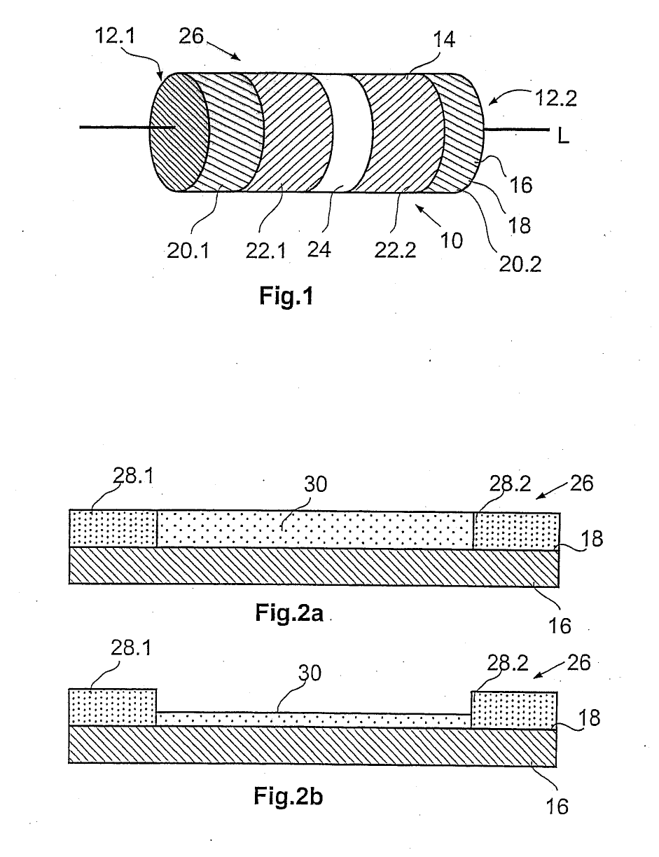

FIG. 1 shows a strongly schematic perspective side view of a stent 10 having a tubular main body 14, which is open at its ends 12.1, 12.2. A peripheral wall 16 of the main body 14, which extends radially around a longitudinal axis L, comprises segments situated neighboring one another in the axial direction, which are in turn assembled from multiple support elements situated in a specific pattern. The individual segments are connected to one another via connection webs and, when assembled, result in the main body 14. In FIG. 1, the illustration of a specific stent design is intentionally dispensed with, since it is not necessary for the purpose of illustrating the present invention and, in addition, it is necessary to individually adapt a coating to the particular geometric factors and other parameters provided for each stent design. Stent designs of greatly varying implementation are known in manifold forms from the related art and will not be explained in greater detail here. It is only to be noted that all current stents 10 have a tubular main frame 14 designed in some way, which comprises a surrounding peripheral wall 16. In the following, an outer mantle surface 18 of the peripheral wall 16 is therefore treated the same as the outer peripheral surface of these support elements, which are possibly formed by multiple existing support elements.

For example, the stent 10 may be molded from a biodegradable magnesium alloy, in particular WE 43. As a result of the transition from its unexpanded state into its expanded state during the dilatation of the stent 10 in the body, the individual support elements are subjected to different mechanical strains, in particular at their joint points. This may result in the metallic structure changing because of microcracking, for example. Typically, especially rapid degradation will occur at points at which an especially high mechanical stress occurs. Furthermore, the individual support elements are dimensioned differently depending on the stent design provided. It is obvious that support elements having a larger circumference are degraded more slowly than corresponding filigree structures in the main frame. The goal for satisfactory degradation behavior of the implant is therefore to counteract a type of splinter formation because of this varying degradation characteristic. The location-dependent degradation characteristic of the main body is expressed in the following in short as D₁(x).

The stent 10 in FIG. 1 shows, in a strongly schematic view, a coating 26, in which multiple sections 20.1, 20.2, 22.1, 22.2, 24 of the outer mantle surface 18 of the peripheral wall 16 are molded from biodegradable materials which are divergent in their degradation characteristic D₂(x). A polymer based on hyaluronic acid is specified here as an example of a suitable material for the coating 26. Hyaluronic acid not only displays a favorable degradation behavior, but rather may also be processed especially easily and additionally has positive physiological effects. The degradation characteristic D₂(x) may be influenced, for example, in such way that a specific degree of cross-linking is predefined by reaction with glutaraldehyde. The higher the degree of cross-linking, the slower will the hyaluronic acid decompose.

Numerous methods have been developed for applying a coating to the stent, such as rotation atomization methods, immersion methods, and spray methods. The coating at least partially covers the wall and/or the individual struts of the stent forming the support structure.

The degradation characteristic D₂(x) differs in the individual sections 20.1, 20.1, 20.2, 22.1, 22.2, 24. Thus—as will be explained in greater detail below—the sections 20.1 and 20.2 at the ends 12.1, 12.2 of the stent 10 may display an accelerated degradation characteristic D₂(x), while in contrast the sections 22.1, 22.2, and 24 situated more in the middle degrade more slowly. In turn, this has the result if one assumes equal degradation characteristic D₁(x) of the main body, degradation occurs more rapidly at the ends of the stent 10. This is advisable because the lesion to be treated is to lie centrally in relation to the sections 22.1, 22.2, and 24 when the stent 10 is applied correctly. Accordingly, the degeneration characteristics D₁(x) and D₂(x) add up to form a cumulative location-dependent degeneration characteristic for the implant.

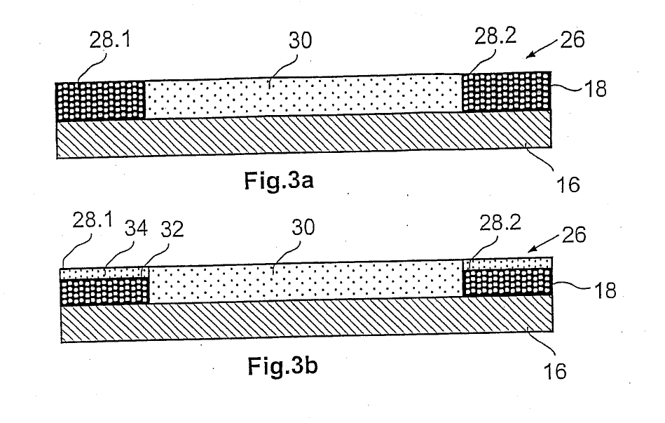

FIGS. 2a, 2b, 3a and 3b show—each in strongly schematic form—a section along the longitudinal axis L of the stent 10, in each case only one of the two sections through the peripheral wall 16 resulting in this case. However, the basic principles in implementing the coating will first be discussed briefly.

A degradation characteristic D₂(x) of a coating at a specific location (x) is essentially a function of factors such as
a layer thickness of the coating,
a morphological structure of the coating, and
a material modification of the coating.

Increasing the layer thickness of the coating lengthens the duration of the degradation. Theoretical and also practical modeling systems have been found which allow estimation of the later in vivo behavior.

Finally, the local degradation characteristic D₂(x) is a function of the morphological structure and material modifications of the coating. Thus, the porosity of the coating may be varied in particular, an increased porosity resulting in accelerated degradation. For material modification, for example, additives may be admixed with the carriers, which delay the enzymatic degradation. A delay of the degradation may also be produced in coatings based on polysaccharide by elevating a degree of cross-linking.

In summary, it is therefore to be noted that by suitably predefining the degradation characteristic D₂(x) of the coating 26, the cumulative degradation characteristic D(x) is predefinable, if the degradation characteristic D₁(x) of the main body is known.

The individual sections of the coating of the stent are also adapted as a function of the pathophysiological and rheological conditions to be expected in application.

The pathophysiological conditions are understood here as the tissue structure changed by illness in the stented vascular area. Typically, the stent is placed in such way that the lesion, i.e., typically the fibrous atheromatotic plaque in coronary applications, is approximately in the middle area of the stent. In other words, the adjoining tissue structures diverge in the axial direction over the length of the stent and therefore a different treatment is also locally indicated under certain circumstances.

The rheological conditions are understood as the flow conditions which result in the individual longitudinal sections of the stent after implantation of the stent. Experience has shown that the ends of the stent have stronger flow around them than the middle areas of the stent. This may result in degradation of the carrier being increased in the end areas.

Too rapid degradation may not support the healing process. Through targeted predefinition of the time interval in which the degradation is to occur at a specific location (x), such incorrect development may be avoided.

Inter alia, all polymer matrices of synthetic nature or natural origin which may be degraded in the living organism on the basis of enzymatic or hydrolytic processes may be used according to the present invention as biodegradable materials for the coating. In particular, polymers from the group comprising cellulose, collagen, albumin, casein, polysaccharides (PSAC), polylactide (PLA), poly-L-lactide (PLLA), polyglycol (PGA), poly-D,L-lactide-co-glycolide (PDLLA/PGA), polyhydroxy butyric acid (PHB), polyhydroxy valeric acid (PHV), polyalkylcarbonates, polyorthoester, polyethylenterephthalate (PET), polymalonic acid (PML), polyanhydrides, polyphosphazenes, polyamino acids and their copolymers, as well as hyaluronic acid, may be used for this purpose. Depending on the desired properties of the coating system, the polymers may be applied in pure form, in derivatized form, in the form of blends, or as copolymers.

If desired, pharmacologically active substances, which are used in particular for treating the results of percutaneous coronary interventions, may be admixed to the coating.

FIG. 2a shows a strongly schematic and simplified sectional view of the peripheral wall 16, having its coating 26 applied to the outer mantle surface 18. The coating 26 comprises two end sections 28.1 and 28.2, as well as a middle section 30. In the present case, the entire coating 26 is formed by a biodegradable material applied in uniform layer thickness.

The sections 28.1, 28.2, 30 differ in that the end sections 28.1, 28.2 degrade more slowly than the middle section 30. This is used in the present exemplary case for compensating for rheological-related accelerations of the degradation process at the stent ends, i.e., the stent schematically illustrated in FIG. 2a will display a degradation behavior which is as homogeneous as possible over the entire length of the stent.

FIG. 2b discloses a second variation of the coating 26. The sections 28.1, 28.2 correspond to those of FIG. 2a. In contrast, the section 30 has its layer thickness significantly reduced. This results in the section 30 being degraded much more rapidly than the sections 28.1 and 28.2. Such a degradation behavior of the implant may be advisable if degradation of the artificial structure is to occur as rapidly as possible in the area of the lesion in order to remove any starting point for possible complications as early as possible in this area.

FIG. 3a shows a coating system 26, in which two different materials having different degradation behaviors are applied in the sections 28.1, 28.2, 30 of the stent 10. This is also true in the variation of the system shown in FIG. 3b.

According to the embodiment shown in FIG. 3a, the sections 28.1, 28.2 are covered by a material having a delayed degradation behavior in relation to the material used in the middle section 30. The location-dependent degradation characteristic D (x) is influenced accordingly, i.e., typically delayed at the ends. Such an embodiment is always advisable if the support structure at the ends is to be maintained over a longer period of time and the rheological conditions otherwise cause an accelerated degradation to be expected.

FIG. 3b shows a multilayered construction of the coating 26 in the radial direction in the sections 28.1 and 28.2. In a first partial section 32, in turn, the material having the delayed degradation behavior is applied, while a partial section 34 having the more rapidly degradable material is located radially outward.

The above-mentioned examples of FIGS. 2a, 2b, 3a and 3b only represent strongly schematic exemplary embodiments of the present invention. They may be combined with one another in manifold ways. Thus, for example, designing a complex coating which comprises multiple materials in individual sections is conceivable. The primary goal is always optimizing the local degradation of the implant in this case.

1. An endovascular implants, comprising:
a) a tubular main body having open front sides and comprising at least one biodegradable material, the main body having a location-dependent first degradation characteristic D₁(x) in vivo; and
b) a coating, which at least partially covers the main body, the coating comprising at least one biodegradable material, the coating having a location-dependent second degradation characteristic D₂(x) in vivo,
wherein a location-dependent cumulative degradation characteristic D(x) results at a location (x) from the sum of the particular existing degradation characteristics D₁(x) and D₂(x) existing at the cited location (x) and the location-dependent cumulative degradation characteristic D(x) is predefined by variation of the second degradation characteristic D₂(x) in such way that the degradation at the cited location (x) of the implant occurs in a predefinable time interval having a predefinable degradation curve.

2. The implant of claim 1, wherein the degradation characteristic D₂(x) of the coating is provided by varying its morphological structure, material modification of the material, or adapting a layer thickness of the coating.

3. The implant of claim 1, wherein the degradation characteristic D₂(x) of the coating is predefined as a function of the pathophysiological conditions to be expected in application.

4. The implant of claim 1, wherein the degradation characteristic D₂(x) of the coating is predefined as a function of the rheological conditions to be expected in application.

5. The implant of claim 2, wherein the degradation characteristic D₂(x) of the coating is predefined as a function of the pathophysiological conditions to be expected in application.

6. The implant of claim 2, wherein the degradation characteristic D₂(x) of the coating is predefined as a function of the pathophysiological conditions to be expected in application.
